(19) United States
(12) Patent Application Publication
(10) Pub. No.: US 2018/0036259 A1
(43) Pub. Date: Feb. 8, 2018

(54) NO DONORS FOR THE TREATMENT OF IMPAIRED TISSUE PERFUSION

(71) Applicant: MIRANDAPHARMACEUTICALS AG, Basel (CH)

(72) Inventors: Pierre VANKAN, Riehen (CH); Hubert STÜCKLER, Graz (AT); Armin SCHERHAG, Dornach (CH)

(73) Assignee: MIRANDAPHARMACEUTICALS AG, Basel (CH)

(21) Appl. No.: 15/552,124

(22) PCT Filed: Feb. 25, 2016
(86) PCT No.: PCT/EP2016/054020

§ 371 Date: Aug. 18, 2017

(30) Foreign Application Priority Data
Feb. 27, 2015 (EP) 15157066.0

(51) Int. Cl. A61K 31/131

(52) U.S. Cl. CPC A61K 31/131; A61K 31/185

(57) ABSTRACT

A method for treatment of prophylaxis of a chronic disease or condition associated with impaired tissue or organ perfusion, comprising administering an amino-C₂-C₆-alkyl nitrate, an amino-C₂-C₄-alkyl nitrate, or a pharmaceutically acceptable salt thereof, to a patient in need thereof. The disease or condition is a micro- and/or macro-vascular disease selected from the group of diseases associated with impaired capillary function, impaired muscular function, impaired skin function, impaired cartilage function, impaired myocardial function, impaired retinal function or impaired organ function caused by impairment of tissue and organ perfusion or by impairment of the regeneration of capillaries, arterioles or other vascular tissue. Examples of such diseases are diseases associated with impaired wound healing (including burning injuries), ulcers (e.g. diabetic foot ulcer), and specifically severe peripheral artery disease including critical limb ischaemia in patients with or without type 2 diabetes, Moyamoya disease or metabolic disorders leading to vascular dysfunction.

The present invention relates to the field of medicine and particularly to the use of exogenous NO donors for the treatment and prophylaxis of a disease or condition associated with impaired tissue and organ perfusion. More particularly the invention provides novel treatment options for diseases with known impairment of macro-, micro and/or capillary perfusion of tissues and/or organs such as peripheral artery disease (PAD), diabetic foot ulcer, angina pectoris, type 2 diabetes mellitus, pulmonary artery hypertension, congestive heart failure, erectile dysfunction, specifically in patients with type 2 diabetes, burning injuries, Moyamoya disease or patients with vascular dysfunction due to metabolic diseases such as MELAS (Mitochondrial Encephalopathy, Lactate Acidosis and Stroke-like symptoms) as a subgroup of mitochondrial diseases.

### BACKGROUND OF THE INVENTION

Currently patients with impaired tissue and organ perfusion are mainly treated with platelet inhibitors like e.g. aspirin or clopidogrel. In the case of peripheral artery disease or other disease with underlying atherosclerosis, current guidelines recommend also the use of statins or vasodilators such as cilostazol or, as an indirect vasodilator, pentoxifyllin. However, the two latter drugs have limited efficacy and the effects of statins may be observed only after several months of treatment. In the case of severe peripheral artery disease with critically reduced perfusion of the legs with pain at rest, interventional treatments like balloon dilatation with or without stent implantation are used. The problem and limitation of these interventional techniques is that they can only be used for relatively large vessels with a diameter of e.g. >1.5 mm. In addition these interventional treatments do as such only improve tissue and organ perfusion indirectly and leave the potential to provide also an improvement of the perfusion in the small arteries (arterioles) and capillaries unused. Given this rationale, a treatment which can improve tissue and organ perfusion on the microvascular level (i.e. in the arterioles and capillaries) or even induce angiogenesis at this microvascular level can be of synergistic and additional use with the other approved treatment approaches described above. A treatment which induces angiogenesis is also expected to be particularly effective in patients wherein microvascular disease, rarefication of arterioles and/or capillaries is the predominant cause for reduced tissue and organ perfusion. This is e.g. the case in patients with type 2 diabetes who suffer often from wound healing problems (e.g. diabetic foot ulcer) in addition to macro- vascular and atherosclerotic peripheral artery disease.

In summary, it can be said that there has been no convincing and long term effective therapeutic approaches for the medical treatment and prophylaxis of chronic diseases or conditions associated with impaired tissue and organ perfusion to date. There is therefore a need for new therapeutic approaches.

### SUMMARY OF INVENTION

The present invention relates to the use of amino-C₂-C₆-alkyl nitrate or a pharmaceutically acceptable salt thereof in the treatment and prophylaxis of a chronic disease or condition associated with impaired tissue or organ perfusion.

It has now been found that amino-C₂-C₆-alkyl nitrate compounds, such as e.g. 2-aminoethyl nitrate or the tosylate salt thereof (CLC-1011), can be used in the treatment and prophylaxis of micro- and/or macro-vascular disease, i.e. a chronic disease or condition associated with impaired tissue or organ perfusion. Moreover it has been found that amino-C₂-C₆-alkyl nitrate compounds, such as e.g. 2-aminoethyl nitrate or the tosylate salt thereof (CLC-1011) do induce the formation of new capillaries and arterioles (angiogenesis) in an animal model of chronic limb ischemia as evidenced in the examples further below. This new and unexpected finding will lead to a significant improvement in the treatment and prophylaxis of a wide variety of chronic diseases or conditions associated with impaired tissue or organ perfusion such as PAD or diabetic foot ulcer.

In particular it has been found that CLC-1011 is not only effective by providing a functional improvement by increasing tissue and organ perfusion by vasodilation, but also by inducing the formation of new blood vessels as shown in the examples further below. The formation of new blood vessels is evidenced by the morphologic changes observed in the treated animals.

It is therefore an object of the present invention to provide new treatment options for the treatment and prophylaxis of a chronic disease or condition associated with impaired tissue and organ perfusion.

The objective is achieved by the finding that certain nitrates can surprisingly be used for the treatment and prophylaxis of a chronic disease or condition associated with impaired tissue and organ perfusion.

Nitrates are well established since a long time in the treatment of acute coronary heart disease and acute myocardial infarction in humans. Such nitrates are however difficult to dose and may induce tolerance after a certain time of use.

As shown in the examples further below amino-C₂-C₆-alkyl nitrates in free base form or in form of a pharmaceutically acceptable salt thereof, in particular 2-aminoethyl nitrate or the tosylate salt thereof (CLC-1011), can be used for the treatment and prophylaxis of a chronic disease or condition associated with impaired tissue and organ perfusion.

Suitable amino-C₂-C₆-alkyl nitrates in free base form or in form of a pharmaceutically acceptable salt thereof have been described in detail in PCT/WO 2014029841 A (CARDIOLYNX AG) 27.02.2014.

Such amino-C₂-C₆-alkyl nitrates are preferably used in the form of an extended release formulation as they are also described in PCT/WO 2014029841. Such pharmaceutical extended release formulation can be administered either in oral or by transdermal means e.g. using a patch in which the active substance or substances are embedded, and which allows sustained release of the active ingredients.

### BRIEF DESCRIPTION OF DRAWINGS

FIG. 1 shows the change in the blood flow over 21 days after treatment with 3 different treatment regimens (large triangles=Cilostazol; diamonds=CLC-1011;small triangles=Cilostazol plus CLC-1011 (N=6/group) vs. placebo (squares). Cilostazol (100 mg/kg) was administered to the animals orally (by gavage, twice daily). CLC-1011 (3.6 mg/day) was administered to the animals via an osmotic pump at 0.25 microliter/hour.

FIG. 2 shows an alternative plot of the data of FIG. 1, viz. the area under the curve for placebo and the treatment arms specified in the legend to FIG. 1. The plot shows that the full effect observed is in essence driven by CLC-1011, because the area under the curve is about equal for Cilostazol and CLC-1011 combined with Cilostazol. The lines above the bars indicate when a difference is statistically relevant.

FIG. 3 shows tissue sections taken after the administration of Cilostazol alone, CLC-1011 alone and the combination of Cilostazol and CLC-1011 vs. placebo. The figure shows a clear increase in the formation of new blood vessels (marked by arrows) in hypoxic limb tissue. This clearly shows that CLC-1011 indeed induces angiogenesis.

FIG. 4 demonstrates the significant arteriogenic effects of CLC-1011. The plot shows the capillary density in the adductor muscle 28 days after surgery after the administration of Cilostazol alone, CLC-1011 alone and the combination of Cilostazol and CLC-1011 vs. placebo (n=4/group).

FIG. 5 demonstrates the significant arteriogenic effects of CLC-1011. The plot shows the arterioles density/mm² in the adductor muscle 28 days after surgery after the administration of Cilostazol alone, CLC-1011 alone and the combination of Cilostazol and CLC-1011 vs. placebo (n=4/group).

FIG. 6 shows the longitudinal blood flow change in ligated hind limb normalized to contralateral limb as assessed by laser Doppler imaging.

FIG. 7 shows in the left panel the arteriolar wall area/luminar area ratio in the four treatment arms (Cilostazol alone, CLC-1011 alone and the combination of Cilostazol and CLC-1011 vs. placebo). The middle panel shows the corresponding wall area (mm²) and the right panel shows the arteriole diameter in mm. FIG. 8 shows the mean fluorescence intensity (MFI) in platelets isolated from the four treatment groups (Cilostazol alone [CILO], CLC-1011 alone and the combination of Cilostazol and CLC-1011 (COMBO) vs. placebo). R=value in the resting state; T=value in the thrombin-activated state.

FIG. 9 shows the circulating SDF-1 levels in serum of rats treated with either placebo, Cilostazol alone, CLC-1011 alone and the combination of Cilostazol and CLC-1011.

FIG. 10 shows the blood flow in diabetic mice after treatment with CLC-1011 or Cilostazol alone or with CLC-1011 and Cilostazol in combination v. placebo. FIG. 10a shows an increased blood perfusion in the mice treated with CLC-1011 alone. FIG. 10b provides the corresponding data plotted as area under the curve.

FIG. 12 shows the capillary density (n/mm²) in the adductor muscles of the mice after treatment with CLC-1011 or Cilostazol alone or with CLC-1011 and Cilostazol in combination v. placebo. In FIG. 11b provides the corresponding data normalized by the number of myocytes counted.

FIG. 12 shows the arterioles density (n/mm²) in the hind limb after treatment with CLC-1011 or Cilostazol alone or with CLC-1011 and Cilostazol in combination v. placebo.

FIG. 13a shows the ratio of arteriolar wall area to luminal area in the hind limb after treatment with CLC-1011 or Cilostazol alone or with CLC-1011 and Cilostazol in combination vs. placebo. FIG. 13b and FIG. 13c provide the corresponding luminal diameter (pm) and wall area (μm²), respectively.

FIG. 14a shows the % cells LinVEGFR2+ in the peripheral blood taken from the mice after treatment with CLC-1011 or Cilostazol alone or with CLC-1011 and Cilostazol in combination vs. placebo. FIG. 14b shows the ° A) cells c-Kit⁺⁻Sca-1 in the peripheral blood taken from the mice after treatment with CLC-1011 or Cilostazol alone or with CLC-1011 and Cilostazol in combination vs. placebo.

### DESCRIPTION OF EMBODIMENTS

The present invention provides amino-C₂-C₆-alkyl nitrates (the therapeutically active ingredient) or a pharmaceutically acceptable salt thereof, for use in the treatment and prophylaxis of a disease or condition associated with impaired tissue and organ perfusion, such as PAD or diabetic foot ulcer.

C₂-C₆-alkyl is an alkyl group consisting of 2 to 6 carbon atoms, in particular ethyl, propyl, butyl, pentyl and hexyl. The alkyl group may be linear, as in n-propyl, n-butyl, n-pentyl and n-hexyl, or branched, as for example in iso-propyl, iso-butyl, sec-butyl, tert-butyl, iso-pentyl, 1-or 2-methylbutyl, 1-ethylpropyl, 1,2-dimethylpropyl, tert-pentyl, and corresponding branched hexyl groups, e.g. iso-hexyl and 1, 2, or 3-methyl-pentyl.

A C₂-C₆-alkyl nitrate is sometimes also called C₂-C₆-alkanol nitrate, indicating more clearly that a nitrate is an ester of nitric acid with the corresponding alkanol, or also a nitrooxyalkane. In the context of the present invention it is understood that in an alkyl nitrate the nitrate function is covalently bonded to the alkyl residue by an oxygen atom.

In amino-C₂-C₆-alkyl nitrate, the amino group and/or the nitrate function may be in a primary, secondary or tertiary position, if possible at all. Preferably, the amino group is not bound to the same carbon atom as the nitrate function. More preferably, both the amino group and the nitrate function are in a primary position, for example as in 2-aminoethyl nitrate, 3-amino-propyl nitrate, 4-aminobutyl nitrate, 5-aminopentyl nitrate, 6-aminohexyl nitrate, 3-amino-2-methylpropyl nitrate, and 3-amino-2,2-dimethylpropyl nitrate. However, other substitution patterns are also considered, for example as in 2-amino-1-methylethyl nitrate, 3-amino-1-methylpropyl nitrate, 2-amino-1, 1-dimethylethyl nitrate, 2-aminopropyl nitrate, 2-aminobutyl nitrate, and 2-amino-3-methylbutyl nitrate.

The said amino-C₂-C₆-alkyl nitrates can be prepared according to methods well known in the art. Most of these amino-C₂-C₆-alkyl nitrates are also commercially available in bulk from various suppliers.

Preferred amino-C₂-C₆-alkyl nitrates are amino-C₂-C₄-alkyl nitrates such as 4-aminobutyl nitrate, 3-aminopropyl nitrate, 2-amino-1-methylethyl nitrate, and 2-aminoethyl nitrate, in particular 4-aminobutyl nitrate and 2-aminoethyl nitrate. Most preferred is 2-aminoethyl nitrate (AEN), also known under the name itramin.

AEN has in the past been marketed by Pharmacia AB under the trade name Nilatil™, but was later again withdrawn from the market. AEN shows an excellent pharmacological profile and in particular does not provoke nitrate tolerance in humans. However, the short half-live of approx. 2 hours requires frequent dosing and causes high peak-to-trough ratios, which is not desirable since it compromises appropriate patient compliance.

Pharmacia has filed for patent protection for certain 2-aminoethylnitrat salts such as e.g. the tosylate salt many decades ago (see SE 168308 C (AB PHARMACIA) 25 Aug. 1959 and U.S. Pat. No. 3,065,136 B (PHARMACIA AB) 20 Nov. 1962).

Pharmaceutically acceptable salts of amino C₂-C₆-alkyl nitrates or amino-C₂-C₄-alkyl nitrates are acid addition salts of pharmaceutically acceptable non-toxic inorganic and organic acids. Preferred pharmaceutically acceptable salts are acetate, benzoate, besylate (benzenesulfonate), bromide, chloride, camphorsulfonate, chlortheophyllinate, citrate, ethenedisulfonate, fumarate, gluconate, glutamate, hippurate, 2-hydroxyethanesulfonate, 2-hydroxy-2-phenylacetate, iodide, lactate, laurylsulfate, malate, maleate, mesylate (methane-sulfonate), methylsulfate, napsylate (2-naphthalenesulfonate), nitrate, octadecanoate, oxalate, pamoate, phosphate, polygalacturonate, succinate, sulfate, tartrate, and tosylate (p-toluenesulfonate).

Most preferred pharmaceutically acceptable salts are sulfate, phosphate, acetate and tosylate, in particular tosylate (e.g. itramin tosilate).

The preferred dosage of the amino-C₂-C₆-alkyl nitrate is between 2 to 8 mg per patient 4 to 5 times daily, with a preferred dosage of 4 mg per patient per dose, when dosed as an immediate release formulation. When dosed as an extended release formulation the dosage is between 5 and 50 mg/once or twice a day or preferably 10 to 25 mg of the active ingredient for a human patient of about 70 kg.

The disease or condition associated with impaired tissue or organ perfusion in accordance with the invention is a disease or condition selected from the group of diseases with impaired capillary function, impaired muscular function, impaired skin function, impaired cartilage function, impaired myocardial function, impaired retinal function or impaired organ function caused by impairment of tissue perfusion, e.g. through impairment of the regeneration of capillaries, arterioles or other vascular tissue.

Examples for a disease or condition associated with impaired tissue and organ perfusion are selected from the group of diseases associated with impaired wound healing (including burn injuries), ulcers, and specifically severe peripheral artery disease (PAD) and critical limb ischaemia in patients with or without type 2 diabetes (e.g. diabetic foot ulcer).

A further example for a disease or condition associated with impaired tissue and organ perfusion is Moyamoya disease (MM). MM is a hereditary neurological disorder that is found predominantly in Japan and South Korea. MM patients suffer from chronic headaches and unclear neurological symptoms caused by transient ischaemic attacks and stroke-like symptoms which are caused by a progressive narrowing of cerebral arteries. To overcome this progressive occlusion of the cerebral vessels, the brain develops a collateral circulation around the narrowed part of the affected cerebral vessel. The newly developed collateral circulation consists of neo-vessels with dysfunctional and dysplastic fragile walls, which can therefore develop into cerebral aneurysms and rupture, resulting in cerebral haemorrhage.

One therapeutic approach for MM is cerebrovascular bypass surgery. This is however a high risk procedure. Otherwise there is currently no effective medical treatment for MM other than general therapeutic approaches with vasodilators and platelet inhibitors.

As noted above nitrate oxide is essential for the maintenance of an intact vascular function. This has been established e.g. in coronary artery disease. In patients with coronary stenosis, a lack of local, vascular NO-production has been described which leads, in addition to the narrowing atherosclerotic plaques in this disease, to local vasoconstriction in the affected vessel regions, further decreasing blood flow and therefore aggravating symptoms. This pathophysiologic cascade can be overcome by administration of organic nitrates. However, their chronic efficacy is compromised by the development of nitrate tolerance.

In MM, it is has been described, that in the affected cerebral vessels there is also a lack of local NO-production, aggravating the perfusion deficits in the depending brain regions, thus leading to progressive symptoms. Therefore, it is concluded that an NO donator without nitrate tolerance will support normal vessel function and will thus delay the development of symptoms by maintaining an intact vascular function as long as possible.

In the case of CLC-1011, chronic NO supplementation with no development of nitrate tolerance will also support the formation of intact cerebral collaterals since NO is known to be an essential factor for angio-neogenesis. CLC-1011 is therefore a unique and promising new treatment option to delay and ameliorate symptoms and disease progression in patients with MM.

A further example for a disease or condition associated with impaired tissue and organ perfusion are Mitochondrial Diseases. Mitochondrial Diseases are a heterogeneous group of genetic disorders in which patients suffer from a lack of energy production from the mitochondria. MELAS (Mitochondrial Encephalopathy, Lactate Acidosis and Stroke-like symptoms) is a subgroup of mitochondrial diseases (approx. 15%). MELAS patients suffer from a wide range of metabolic complications, including oxidative stress and vascular NO deficiency causing migraine-like symptoms, seizures, strokes and delayed mental development.

Currently, there is beyond general measures, such as avoidance of stress, supplementation of vitamins and co-enzyme, no specific treatment for Mitochondrial Diseases and in particular MELAS patients. Because CLC-1011 is bio-activated via an extra mitochondrial pathway (most likely via xanthine oxidase), it provides a novel unique treatment option to supply NO and thus alleviate the metabolic and vascular NO deficiency.

In another embodiment the invention provides an amino C₂-C₆-alkyl nitrate or an amino C₂-C₄-alkyl nitrate or a pharmaceutically acceptable salt thereof, for use in the treatment and prophylaxis of a chronic disease or condition associated with impaired tissue or organ perfusion, wherein the amino-alkyl nitrate is in the form of an extended release composition. Preferably the extended release composition is administered once or twice daily. The preferred dosage range is between 5 and 50 mg/once or twice a day.

The preparation of extended release formulations of C₂-C₆-alkyl nitrate and in particular 2-aminoethyl nitrate (AEN) for the treatment of a variety of diseases such as inter alia Morbus Raynaud and Morbus Meniére is described in PCT/WO 2014029841 A (CARDIOLYNX AG) 27.02.2014.

In another embodiment the invention provides 2-aminoethyl nitrate or the tosylate salt thereof (CLC-1011) as an immediate release formulation for use in the treatment and prophylaxis of peripheral artery disease, angina pectoris, type 2 diabetes mellitus, pulmonary artery hypertension, diabetic foot ulcer, congestive heart failure, erectile dysfunction, specifically in patients with type 2 diabetes, burning injuries, Moyamoya disease or patients with vascular dysfunction due to metabolic diseases such as MELAS as a subgroup of mitochondrial diseases.

In another embodiment the invention provides 2-aminoethyl nitrate or the tosylate salt thereof (CLC-1011) as an extended release formulation for use in the treatment and prophylaxis of peripheral artery disease, diabetic foot ulcer, angina pectoris, type 2 diabetes mellitus, pulmonary artery hypertension, diabetic foot ulcer, congestive heart failure, erectile dysfunction, specifically in patients with type 2 diabetes, burning injuries, Moyamoya disease or patients with vascular dysfunction due to metabolic diseases such as MELAS as a subgroup of mitochondrial diseases.

The term “extended release” as used herein means that the release of the active ingredient does not occur by immediate release, but represents release over a pre-defined, longer time period of time, such as e.g. of up to 24 hours, such as e.g. 4 to 24, 6 to 24 hours, or preferably 12 to 24 hours. Release characteristics and release time are measured according to standard methods, e.g. those of Ph. Eur., in aqueous solution with a paddle at pH 6.8 and 37° C.

The extended-release characteristics for the release of amino-C₂-C₆-alkyl nitrate or the amino-C₂-C₄-alkyl nitrate and pharmaceutically acceptable salts thereof may be varied by modifying the composition of each formulation component, including modifying any of the excipients and coatings or also transdermal patch layers which may be present. In particular the release of the active ingredient may be controlled by changing the composition and/or the amount of the extended-release coating, if such a coating is present. If more than one extended-release component is present, the coating or matrix former for each of these components may be the same or different. Similarly, when extended-release is governed by an extended-release matrix material, release of the active ingredient may be controlled by the choice and amount of extended-release matrix material utilized.

When the extended-release component comprises a modified release matrix material, any suitable extended-release matrix material or suitable combination of extended-release matrix materials may be used. Such materials are known to those skilled in the art. Thus e.g. polymer coated drug-ion exchange resins comprising a very large variety of known drugs, such as e.g. itramin, are described in PCT/WO 2008064163 A (MORTON GROVE PHARMACEUTICALS, INC) 29 Feb. 2008. The term “ extended-release matrix material” as used herein includes hydrophilic polymers, hydrophobic polymers and mixtures thereof which are capable of modifying the release of an active ingredient dispersed therein in vitro and in vivo.

In a preferred embodiment, the extended-release composition for use in the treatment and prophylaxis of a disease or condition associated with impaired tissue or organ function in accordance with the present invention will provide more or less constant plasma levels of amino-C₂-C₆-alkyl nitrate or amino-C₂-C₄-alkyl nitrate and the pharmaceutically acceptable salts thereof over 12 hours, more preferably over 24 hours.

One of the objects of this invention is therefore to provide an extended release oral dosage form for use in the treatment and prophylaxis of a disease or condition associated with impaired tissue or organ perfusion. In such an extended release oral dosage form the active ingredient or ingredients may be embedded in a non-ionic polymer matrix, which matrix may optionally be coated.

For oral dosage forms, any coating material which modifies the release of the active ingredient in the desired manner may be used. In particular, coating materials suitable for use in the practice of the invention include but are not limited to polymer coating materials, such as cellulose acetate phthalate, cellulose acetate trimellitate, hydroxy propyl methylcellulose phthalate, polyvinyl acetate phthalate, ammonium methacrylate copolymers such as those sold under the trademark Eudragit™ RS and RL, polyacrylic acid and polyacrylate and methacrylate copolymers such as those sold under the trademark Eudragit™ S and L, polyvinyl acetaldiethylamino acetate, hydroxypropyl methylcellulose acetate succinate, shellac; hydrogels and gel-forming materials, such as carboxyvinyl polymers, sodium alginate, sodium carmellose, calcium carmellose, sodium carboxymethyl starch, polyvinyl alcohol, hydroxyethyl cellulose, methyl cellulose, gelatin, starch, and cellulose based cross-linked polymers in which the degree of crosslinking is low so as to facilitate adsorption of water and expansion of the polymer matrix, hydoxypropyl cellulose, hydroxypropyl methylcellulose, polyvinyl pyrrolidone, crosslinked starch, microcrystalline cellulose, chitin, aminoacryl-methacrylate copolymer (Eudragit™ RS-PM), pullulan, collagen, casein, agar, gum arabic, and sodium carboxymethyl cellulose.

In a particular embodiment of the extended release dosage form in accordance with the present invention, a non-ionic matrix material is used.

As will be appreciated by the person skilled in the art, excipients such as plasticisers, lubricants, solvents and the like may be added to the coating. Suitable plasticisers include, for example, acetylated monoglycerides, butyl phthalyl butyl glycolate, dibutyl tartrate, diethyl phthalate, dimethyl phthalate, ethyl phthalyl ethyl glycolate, glycerol, propylene glycol, triacetin, citrate, tripropionin, diacetin, dibutyl phthalate, acetyl monoglyceride, polyethylene glycols, castor oil, triethyl citrate, polyhydric alcohols, acetyl triethyl citrate, dibenzyl phthalate, dihexyl phthalate, butyl octyl phthalate, di-isononyl phthalate, butyl octyl phthalate, dioctyl azelate, epoxidized tallate, tri-isoctyl trimellitate, diethylhexyl phthalate, di-n-octyl phthalate, di-isooctyl phthalate, di-isodecyl phthalate, di-n-undecyl phthalate, di-n-tridecyl phthalate, tri-2-ethylhexyl trimellitate, di-2-ethylhexyl adipate, di-2-ethylhexyl sebacate, di-2-ethylhexyl azelate, and dibutyl sebacate.

For oral dosage forms, extended release of the active ingredient in the desired manner may be obtained by embedding the drug in a matrix. In matrix devices, the active agent appears as dispersion within the polymer matrix and is typically formed by the simple compression of a polymer/drug mixture, through its dissolution in a common solvent or melt granulation. Matrix formulations are particularly preferred for controlling the release of the active ingredient of the present invention, since they are relatively easy to manufacture compared to other devices.

Among the many oral dosage forms that can be used for extended drug release, matrix tablets, as obtained by the direct compression of a polymer mixture, are preferred. Matrix materials considered are biocompatible natural polymers, e.g. HPMC, HEMC, EHEC, HMHEC, CMHEC, methylcellulose, guar, pectin, agar, algin, gellan gum, xanthan gum, acacia, starch and modified starches, carrageenans, amylose starch, and the like.

Particular swellable hydrophilic polymers considered as matrix materials are poly(hydroxyalkanol methacrylate) (MW 5 kD-5,000 kD), polyvinylpyrrolidone (MW 10 kD-360 kD), anionic and cationic hydrogels, polyvinyl alcohol having a low acetate residual, a swellable mixture of agar and carboxymethyl cellulose, copolymers of maleic anhydride and styrene, ethylene, propylene or isobutylene, pectin (MW 30 kD-300 kD), polysaccharides such as agar, acacia, karaya, tragacanth, algins and guar, polyacrylamides, Polyox™ polyethylene oxides (MW 100 kD-5,000 kD), AquaKeep™ acrylate polymers, diesters of polyglucan, crosslinked polyvinyl alcohol and poly N-vinyl-2-pyrrolidone, and sodium starch glucolate (e.g. Explotab™; Edward Mandell Co. Ltd.).

Further particular hydrophilic polymers considered are polysaccharides, methyl cellulose, sodium or calcium carboxymethyl cellulose, hydroxypropyl methyl cellulose, hydroxypropyl cellulose, hydroxyethyl cellulose, nitro cellulose, carboxymethyl cellulose, cellulose ethers, polyethylene oxides (e.g. Polyox™, Union Carbide), methyl ethyl cellulose, ethyl hydroxylethylcellulose, cellulose acetate, cellulose butyrate, cellulose propionate, gelatin, collagen, starch, maltodextrin, pullulan, polyvinyl pyrrolidone, polyvinyl alcohol, polyvinyl acetate, glycerol fatty acid esters, polyacrylamide, polyacrylic acid, copolymers of methacrylic acid or methacrylic acid (e.g. Eudragit™), other acrylic acid derivatives, sorbitan esters, natural gums, lecithins, pectin, alginates, ammonium alginate, sodium, calcium and/or potassium alginates, propylene glycol alginate, agar, and gums such as arabic, karaya, locust bean, tragacanth, carrageens, guar, xanthan, scleroglucan and mixtures and blends thereof.

Preferred modified release matrix materials suitable for the practice of the present invention are microcrystalline cellulose, sodium carboxymethylcellulose, hydoxyalkylcelluloses such as hydroxypropylmethylcellulose and hydroxypropylcellulose, polyethylene oxide, alkylcelluloses such as methylcellulose and ethylcellulose, polyethylene glycol, polyvinylpyrrolidone, cellulose acteate, cellulose acetate butyrate, cellulose acteate phthalate, cellulose acteate trimellitate, polyvinylacetate phthalate, polyalkylmethacrylates, polyvinyl acetate, and mixture thereof.

Another object of the invention is to provide a multiparticulate extended release composition for use in the treatment and prophylaxis of a disease or condition associated with impaired tissue or organ perfusion.

The term “particulate” as used herein refers to a state of matter which is characterized by the presence of discrete particles, pellets, beads, granules, or small tablets (“mini-tablets”) irrespective of their shape or morphology.

The term “multiparticulate” as used herein means a plurality of discrete or aggregated particles, pellets, beads, granules, small tablets or mixture thereof irrespective of their shape or morphology. The term “multiparticulate” includes every subunit of a size smaller than 5 mm, e.g. pellets, granules, sugar seeds (non-pareil), minitablets, powders, and crystals, with drugs being entrapped in or layered around cores.

Although similar in vitro drug release profiles can be obtained with single-unit and multiple-unit dosage forms, the latter offer several advantages over single-unit systems such as non-disintegrating tablets or non-disintegrating capsules, and represent a preferred embodiment of the invention.

Multiparticulates in accordance with the present invention are filled into capsule or compressed into tablets. Tablets allow inserting a breaking score, making it possible to sub-divide the dose, and still maintaining the extended-release characteristics of the multiparticulate formulation. Multiparticulates provide many advantages over single-unit systems because of their small size. They are less dependable upon gastric emptying, resulting in less inter- and intra-subject variability in gastrointestinal transit time. They are also better distributed and less likely to cause local problems. Other advantages include adjustment of the strength of a dosage unit by changing the number of multiparticulates in the unit, administration of incompatible drugs in a single dosage unit by separating them in different multiparticulates, and combination of multiparticulates with different drug release rates to obtain the desired overall release profile. In multiple unit systems, the total drug dose is divided over multiparticulates that make up that system. Failure of a few units may not be as consequential as failure of a single-unit system, where a failure may lead to dose-dumping of the drug.

Still another object of the present invention is to provide a single-unit extended release tablet-, film coated tablet- or hard capsule formulation of an amino-C₂-C₆-alkyl nitrate or an amino-C₂-C₄-alkyl nitrate and the pharmaceutically acceptable salts thereof, for use in the treatment and prophylaxis of a disease or condition associated with impaired tissue or organ perfusion. The term “hard capsules” includes any type of hard capsule made from gelatine or a different material, e.g. hypromellose and gellan gum (Vcaps™) or pullulan and carrageenan (NPcaps™).

Another object of the present invention is to provide an osmotically controlled oral dosage form of an amino-C₂-C₆-alkyl nitrate or an amino-C₂-C₄-alkyl nitrate and the pharmaceutically acceptable salts thereof, for use in the treatment and prophylaxis of a disease or condition associated with impaired tissue or organ perfusion. Drug delivery from an osmotic drug delivery system is not influenced by the different physiological factors within the gut. Osmotic drug delivery systems (ODDS), apart from maintaining plasma concentration within therapeutic range, also prevent sudden increase in plasma concentration that may produce side effects and sharp decrease in plasma concentrations that may reduce the efficacy of the drug. Considered are single-layer core osmotic pumps using conventional tablets as a core. Elementary osmotic pump (EOP) and the controlled porosity (OP) are two different embodiments of this technology. Further preferred are dosage forms based on the so-called “push-pull system” using a bi-layer kernel (multi-layer core osmotic pumps).

Another object of the present invention is to provide an amino C₂-C₆-alkyl nitrate or an amino C₂-C₄-alkyl nitrate or a pharmaceutically acceptable salt thereof, for use in the treatment and prophylaxis of a disease or condition associated with impaired tissue or organ perfusion, wherein the amino-alkyl nitrate is in the form of a transdermal drug delivery system. Such transdermal drug delivery system allows the release of the active ingredient continuously over a time period of several hours up to several days. Preferably the transdermal drug delivery system releases the active ingredient in accordance with the present invention continuously over a time window of approximatively 12 to 24 hours. Alternatively, the transdermal drug delivery system releases the active ingredient in accordance with the present invention continuously over a time window of 1 to 7 days.

Another object of the present invention is to provide transdermal patches wherein the amino-alkyl nitrate in accordance with the present invention is embedded and where upon administering to the skin the therapeutically active ingredient is released over an extended period of time, e.g. within 24 hours and taken up by the body through the skin.

The skin permeation rate of amino-C₂-C₆-alkyl nitrates or amino-C₂-C₄-alkyl nitrates are positively influenced by chemical enhancers, for example co-solvents such as ethanol, isopropanol, glycerol, polyethylene glycol, propylene glycol, pyrrolidones, dimethylsulfoxide, laurocapram (1-dodecylazepan-2-one) and the like, surfactants, fatty acid esters such as polyethylene glycol monolaurate, and terpenes such as menthol. Preferred dermal penetration enhancers are laurocapram and laurocapram derivatives, and oleic acid and its esters, such as methyl, ethyl, propyl, isopropyl, butyl, vinyl and glyceryl esters, dodecyl (N,N-dimethylamino)acetate and dodecyl (N,N-dimethylamino)propionate, and 2-n-nonyl-1-3-dioxolane. Most preferred dermal penetration enhancers are oleic acid and its esters, dodecyl (N,N-dimethylamino)-acetate and -propionate, and 2-n-nonyl-1-3-dioxolane. The penetration enhancers facilitate the delivery of drugs through the skin by temporarily altering the top skin barrier layer.

A variety of transdermal technologies, including reservoir patches, matrix patches, poration devices and iontophoretic devices are considered. Preferred are drug-in-adhesive and reservoir patches. The drug-in-adhesive patches combine adhesive and the drug in a single layer making it less costly to manufacture. Such patches may be used for several days, e.g. up to 7 days, are light weight and thin, and can be rather small and translucent.

Another object of the present invention is to provide drug-in adhesive patches wherein the amino-alkyl nitrate for use in accordance with the present invention is embedded and where upon administering to the skin the therapeutically active ingredient is released over an extended period of time, e.g. within 12 to 24 hours and taken up by the body through the skin.

Drug-in-adhesive systems incorporate amino-C₂-C₆-alkyl nitrates or amino-C₂-C₄-alkyl nitrates into a carrier such as a polymeric matrix and/or a pressure-sensitive adhesive, such as silicone adhesive, silicone rubber, acrylic adhesive, polyethylene, polyisobutylene, polyvinyl chloride, nylon, or the like formulation. The pressure-sensitive adhesive must adhere effectively to the skin and permit migration of the active ingredient from the carrier through the skin and into the bloodstream of the patient. The amino-C₂-C₆-alkyl nitrate or amino-C₂-C₄-alkyl nitrate is directly incorporated into the adhesive in one single layer, or alternatively dissolved in the polymeric matrix until its saturation concentration is reached. When drug migrates through the skin and thereby is removed from the surface of the matrix, more of the drug diffuses out of the interior in response to the decreased concentration at the surface. The release rate is therefore not constant over time, but instead gradually decreases as the drug concentration decreases.

Another object of the present invention is to provide reservoir patches wherein the amino-alkyl nitrate for use in accordance with the present invention is embedded and where upon administering to the skin the therapeutically active ingredient is released over an extended period of time, e.g. within 12 to 24 hours and taken up by the body through the skin.

Reservoir patches contain a reservoir or a pocket which holds the amino-C₂-C₆-alkyl nitrate or amino-C₂-C₄-alkyl nitrate, encapsulated in a gel. A protective seal covers the contents of the patch. A permeable film allows the nitrates to flow through at a controlled rate.

In order to modify the rate of delivery from the transdermal device, a specific single-polymer matrix or a blend of soluble (miscible) polymers is considered. Polymers considered are those listed above for oral drug forms.

The drug-in-adhesive patches are manufactured by following sequence of steps: Appropriate amounts of adhesives are dissolved in a solvent in a vessel. The amino-C₂-C₆-alkyl nitrate or the amino-C₂-C₄-alkyl nitrate or the pharmaceutically acceptable salt thereof, respectively, is added and dissolved/dispersed in a polymer mixture, and optional co-solvents and enhancers are added. The solution is coated onto a protective release liner at a controlled specified thickness. Then the coated product is passed through an oven to drive off volatile solvents. The dried product on the release liner is combined with the backing material and wound into rolls for storage. The roll of film is then cut at the desired size and packaged.

Another object of the present invention is to provide pharmaceutical compositions comprising an amino-alkyl nitrate in accordance with the present invention for the treatment and prophylaxis of a disease or condition associated with impaired tissue and organ perfusion, such as e.g. PAD or diabetic foot ulcer.

Another object of the present invention is the use of an amino-alkyl nitrate in accordance with the present invention for the treatment and prophylaxis of a disease or condition associated with impaired tissue and organ perfusion, such as e.g. PAD or diabetic foot ulcer.

Another object of the present invention is the use of an amino-alkyl nitrate in accordance with the present invention for the manufacture of a medicament for the treatment and prophylaxis of a disease or condition associated with impaired tissue and organ perfusion, such as e.g. PAD or diabetic foot ulcer.

The present invention will now be described more fully by the examples provided herein below. The present invention may, however, be embodied in different forms and the examples below should not be construed as limiting the embodiments set forth herein. Rather, these examples are provided so that this disclosure will be thorough and complete, and will fully convey the scope of the invention to those skilled in the art.

### EXAMPLES

The primary goal of the study was to investigate the effects of CLC-1011 on blood perfusion in an animal model of periphery arterial disease (PAD). There is an absolute unmet medical need to treat growing population suffering from PAD. In addition the currently widely used drug to treat PAD, viz. Cilostazol, has been criticized by the regulatory agencies due to its adverse effects and limited efficacy.

### Example 1

The Sprague-Dawley (SD) Rats Used for the Following Experiments were 5 weeks of age, male, and had a mean weight of approximately 100 g. The animals were held in the Open Animal Facility at the Department of Physiology, University of Zurich and used after a suitable period allowing the animals to adapt to the new environment. A unilateral hind limb ischemia was created in the SD rats by surgical intervention, whereby the right hind limb was ligated and the vessels excised as described in Vasc. Pharmacol., 2009, 51(4):268-74. The animals were then randomly assigned to receive either Cilostazol alone (100 mg/kg/bi-daily orally by gavage), CLC-1011 (3.6 mg/day by mini-osmotic pump, e.g. Model #2004, Alzet Inc.) alone, a combination of Cilostazol (100 mg/kg/bi-daily) and CLC-1011 (3.6 mg/day by mini-osmotic pump), or placebo for a total duration of 2 or 4 weeks.

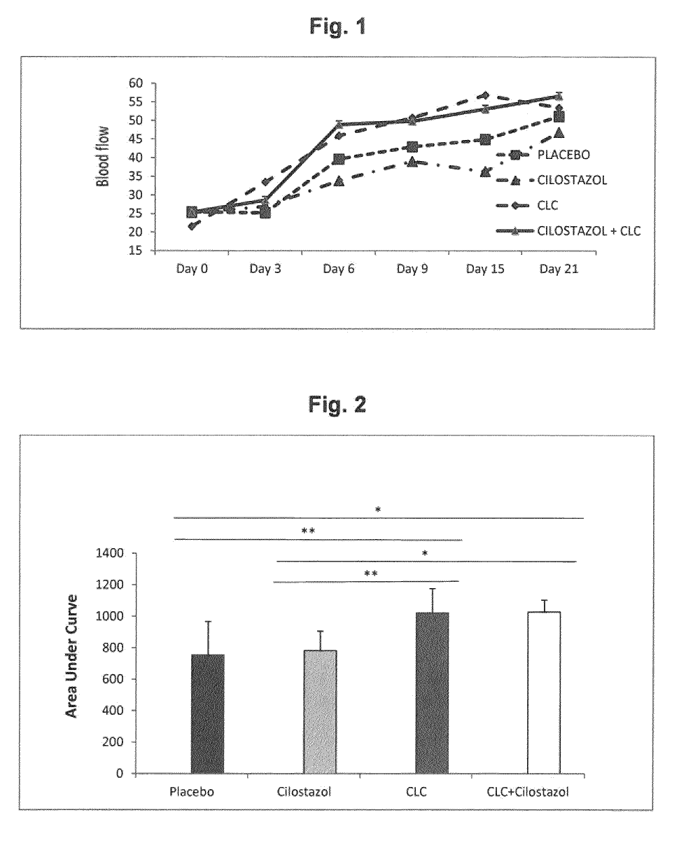

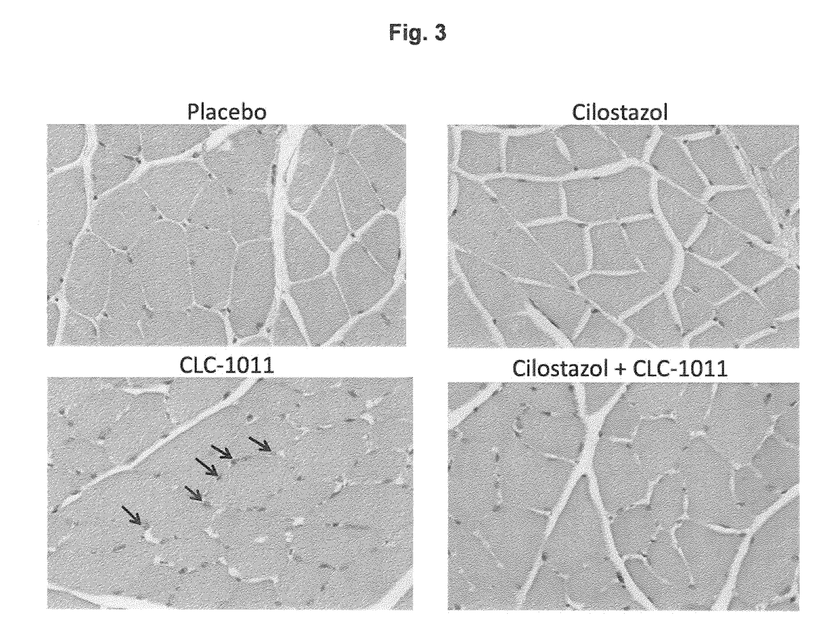

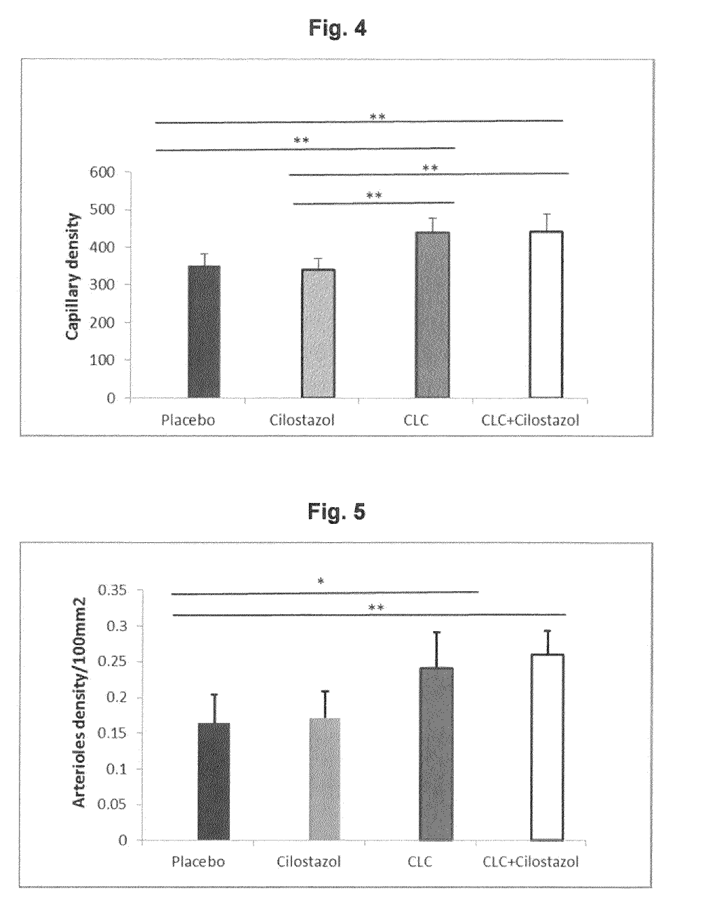

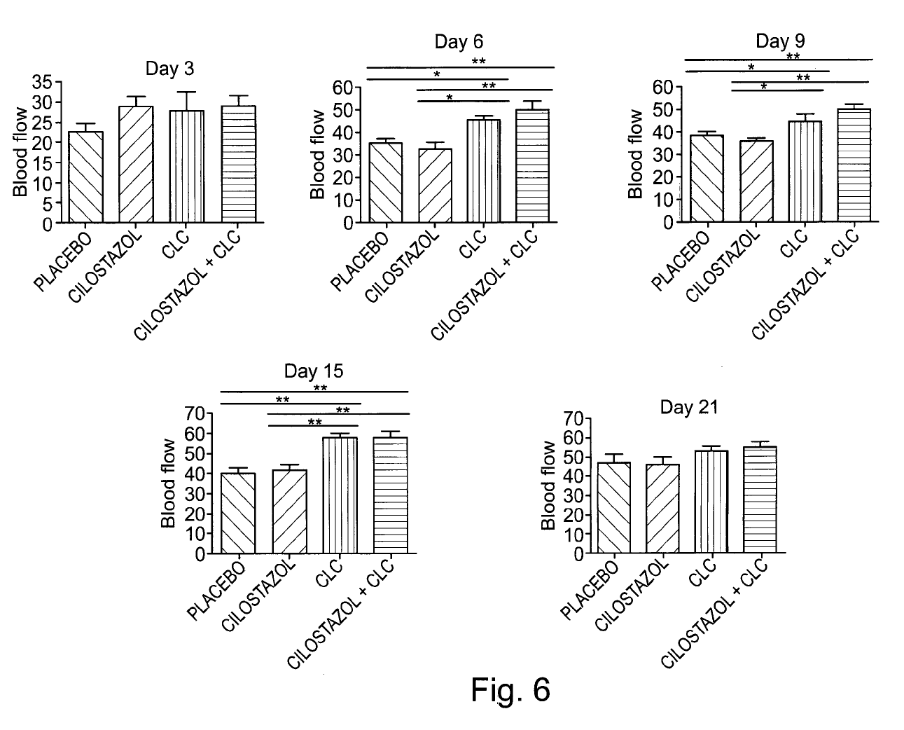

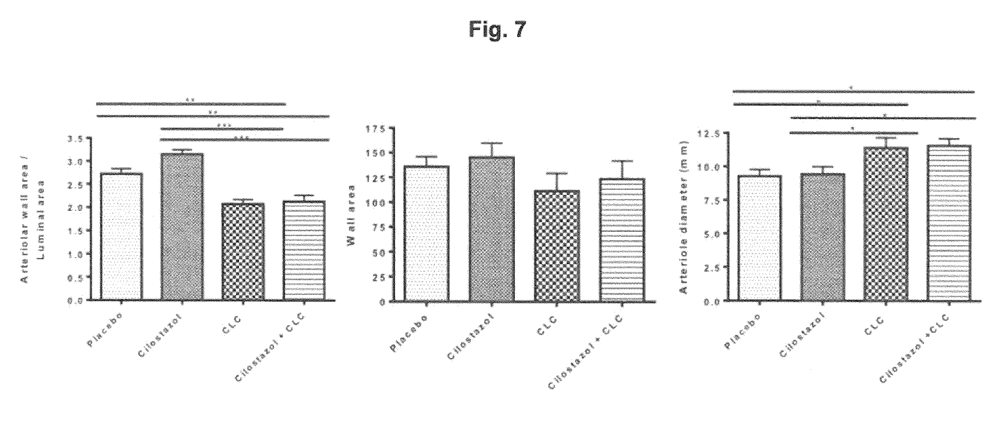

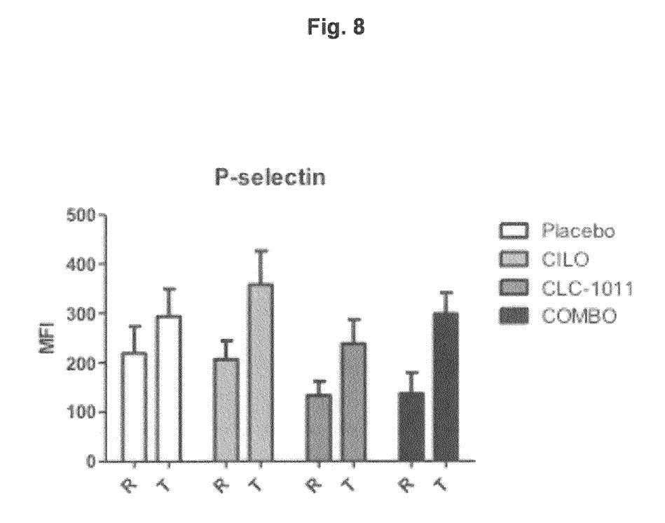

During the treatment intervals, animals were scanned using laser Doppler imaging every third day post-operation to estimate the blood perfusion in ligated limbs. Blood flow was measured using a laser Doppler imager (Moor, Axminster, UK) and analyzed with the MoorLDI™ Image Review V51 software. The animals were placed on a heating pad in order to maintain constant body temperature during the entire procedure. At the end of the 2 and 4 weeks interval, animals were sacrificed and muscles were isolated and prepared for histological analysis. The results presented herein indicate an increase in blood perfusion in the CLC-1011-treated arm, whereas no effect in blood flow was seen using Cilostazol alone (FIG. 1). No statistical relevant additional effect was seen when CLC-1011 was combined with Cilostazol (see FIG. 2). The histological analysis showed that there is an increase in the formation of new blood vessels in the hypoxic limb tissue after treatment with CLC-1011 (see arrows in FIG. 3). The histological data also indicate that angiogenesis has been stimulated in CLC-1011-treated group in the muscles of animals after 2 and 4 weeks of treatment (see FIG. 4 showing the significant effects on adductor muscle capillarisation 28 days after surgery). Similarly, CL-1011 significantly contributes to an increase of the number of arterioles in isolated muscles as compared to Cilostazol- and placebo-treated animals, at 4 weeks (FIG. 5). The increase in blood perfusion can be detected up to 3 weeks when the difference in increased perfusion cannot be detected any longer using laser Doppler imaging (see FIG. 6 showing the longitudinal blood flow change in ligated hind limb normalized to contralateral limb).

### Example 2

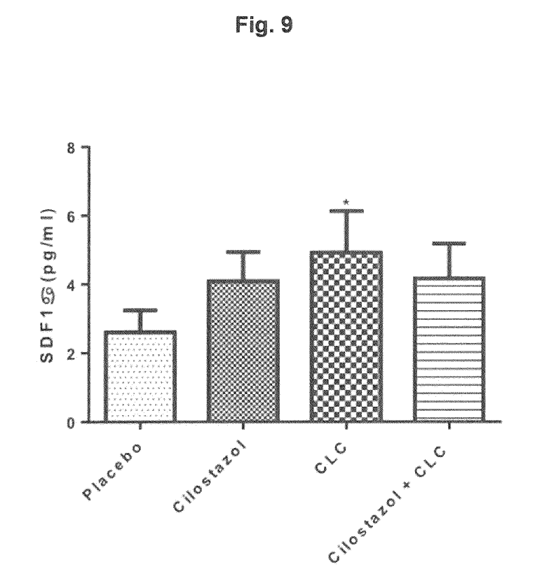

In order to assess possible effects the different treatments have on vascular remodelling, histopathological analysis of isolated adductor muscle was performed and the vascular luminal- and vascular wall-properties were addressed. It was found that treatment with CLC-1011 significantly increases luminal area of the vessels as compared to Cilostazol and placebo treatments, while such effect is absent in placebo- and Cilostazol-treated SD rats. See FIG. 7 showing in the left panel the arteriolar wall area/luminar ratio in the four treatment arms. The middle panel shows the results for the wall area (mm²) and the right panel the results of the arteriole diameter (mm).

### Example 3

Platelet activation was determined using mean fluorescence measurements. It was found that CLC-1011 significantly prevents activation of platelets in resting state, while Cilostazol has no effect in prevention of platelet activation and aggregation. FIG. 8 shows the measured values of the mean fluorescence intensity in platelets isolated from the four treatment groups (R resting state; T=thrombin-activated state).

### Example 4

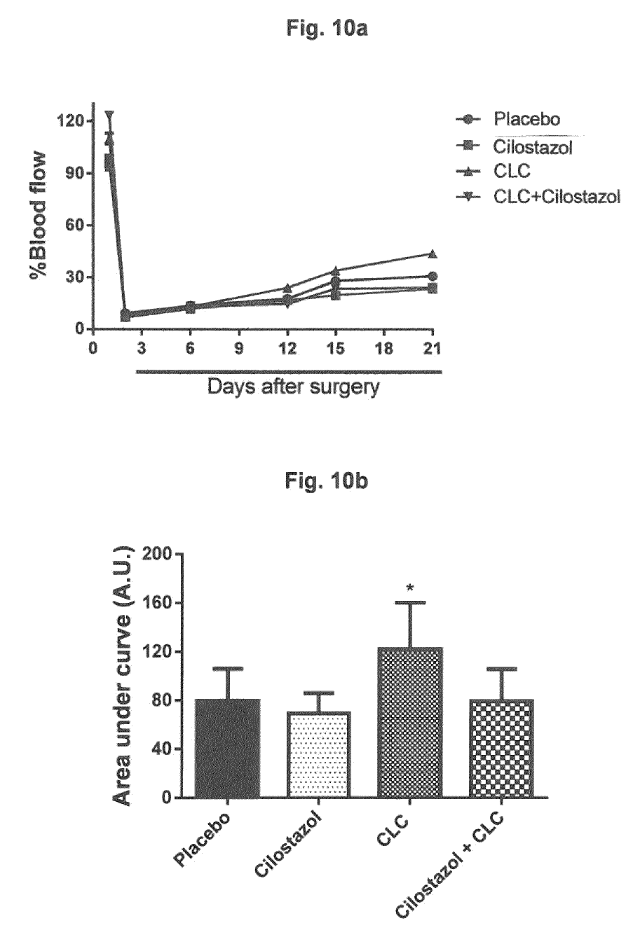

The stromal cell-derived factor 1 (SDF-1) is a circulating proangiogenic cytokine, which plays an important role in angiogenesis. Serum of rats from 4 treatment arms was collected at day 6 post-ischemia. SDF-1 levels were determined by ELISA using methods known in the art. It was found that CLC-1011-induced the release of the circulating SDF-1 (FIG. 9).

### Example 5

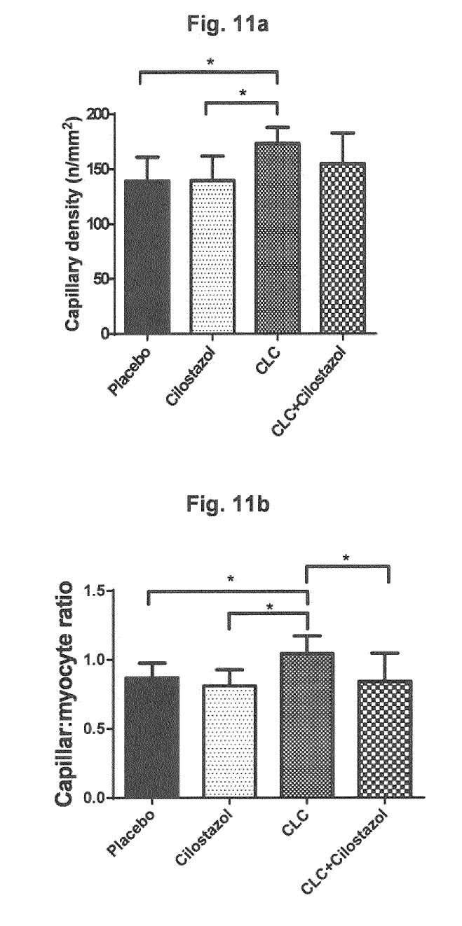

This example shows the superior properties of CLC-1011 in an experimental hind limb model in a diabetic mouse model (Bks.Cg+Lepr<db>/+Lepr<db>/OlaHsd). Blood flow was measured by laser Doppler imaging in diabetic mice with hind limb ischemic after treatment with CLC-1011 alone or in a combination with Cilostazol. The results show an increase in the blood perfusion only in the mice treated with CLC-1011(FIG. 10a), while FIG. 10b indicates an increase of the area under the curve in CLC-1011-treated arm only. Blood perfusion in placebo-, combination-, or Cilostazol-only- treated animals remains unchanged.

Immunohistochemical analysis of the adductor muscles showed an increase of the capillary density only in CLC-1011-treated animals (FIG. 11a) normalized by the number of myocytes counted (FIG. 11b) indicating CLC-1011-driven increase of the capillaries while the myocyte count remain unchanged in all treatment arms.

Immunohistology for the alpha-smooth muscle (α-SMA) antigen was employed to highlight the vascular structures within the muscles and allow accurate counting of arterioles. Briefly, sections were deparaffinised in xylene (2×5 min) and rehydrated in decreasing concentrations of ethanol (2×3 min washes in 100% ethanol, followed by 1×3 min wash in 96% ethanol). Sections were washed twice with Tris-buffered saline (TBS, 0.1 M Tris- HCl with 0.9% NaCl, pH 7.4) and incubated for 1 h at room temperature (RT) with the primary antisera (M0851, Dako, Baar, Switzerland), without pretreatment. A detection kit containing the secondary antibody and diaminobenzidine tetrahydrochloride (DAB) as chromogen was subsequently applied according to the manufacturer's protocols

(Peroxidase/DAB plus Mouse Kit; DAKO). Sections were then washed 3 times in TBS and once in distilled water and counterstained for 1 min with haematoxylin, followed by rinsing for 5 min in tap water and dehydration in ascending alcohols, clearing in xylene, coverslipping and mounting. Arterioles density was quantified in histological sections of the adductor muscle. Results were expressed as number of arterioles/mm² of tissue.

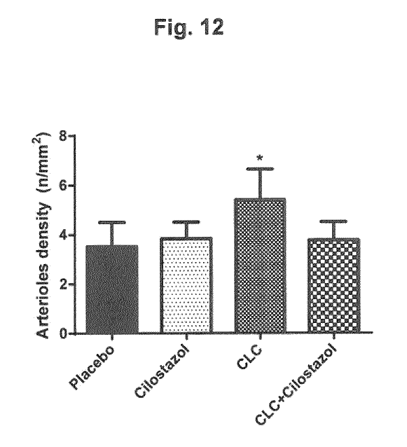

The results demonstrated an increase of the arterioles in the hind limb of animals treated with CLC-1011 while no change was observed in placebo-, combination-, or Cilostazol-only- treated animals (FIG. 12).

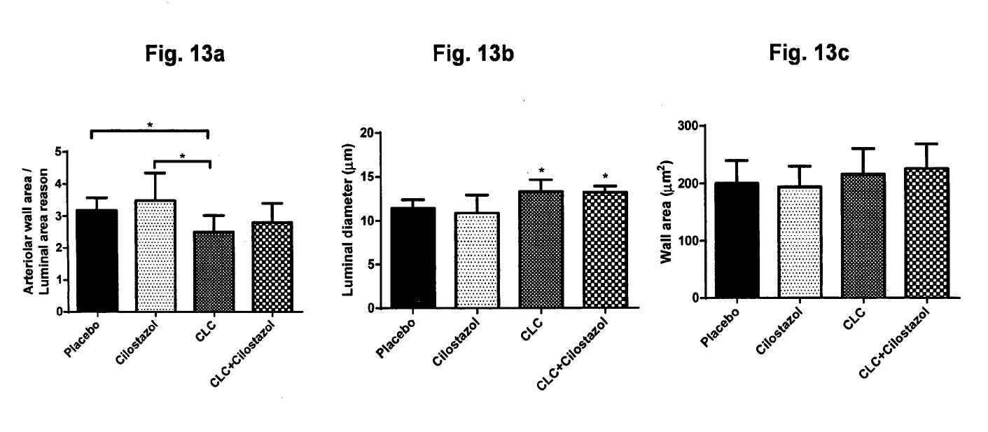

These findings corroborate with the remodeling effect of CLC-1011 seen at the vascular wall. To check the arteriolar remodeling response to the ischemia, immunohistochemistry was performed as described above. The remodeling consists in the wall area per luminal area ratio. The measurement of wall and luminal areas and diameter calculation of arterioles was performed in an equal random area for each histological sample. The results shown in FIG. 13a; FIG. 13b and FIG. 13c indicate that the luminal area of the vessels increased in the CLC-1011-treated animals, but not in the Cilostazol- or the placebo-treated animals. Of note, the results indicated no change in the wall area size in either of the groups.

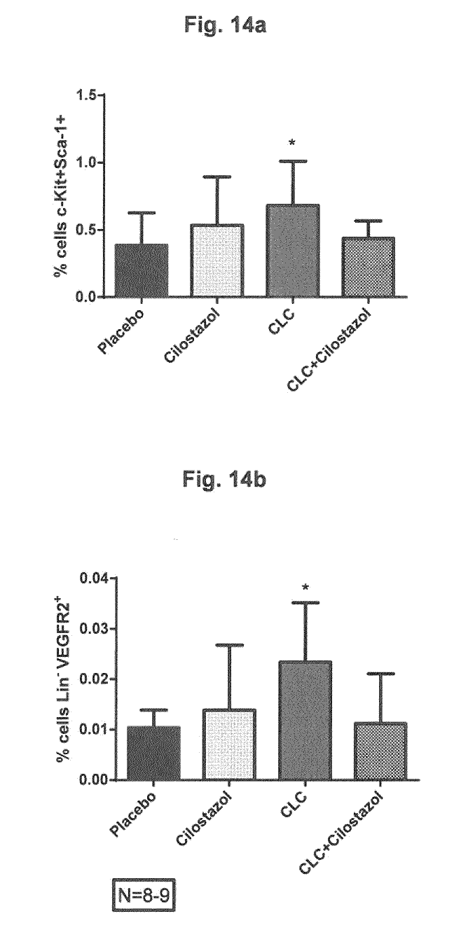

Moreover it was observed that CLC-1011 not only increases the blood flow in the ligated limb, but also enhances the recruitment of the endothelial progenitor cells. Mobilization of putative stern cells from bone marrow into the peripheral blood in the placebo and treated groups mice 7 days after surgery was assessed by flow cytometry in whole blood. The cells were identified using a combination of antibodies that binds to the specific cell surface markers typical for potential angiogenic stem cells. The data in FIG. 14 indicate an increase in the number of potential endothelial progenitor cells which are positive for the stem cell antigen 1 (Sca-1) (FIG. 14a) as well as for the vascular endothelial growth factor receptor 2 (VEGFR2) (FIG. 14b) only in CLC-1011-treated animals. Since Sca-1 is postulated to have an important regenerative role following myocardial infarction and VEGFR2 is an important receptor for signaling in both vasculogenesis and angiogenesis, our data indicate that the angiogenic and arteriogenic response is promoted by CLC-1011 at the site of the ischemic injury.

The data of shown in Example 5 support the importance of the further clinical development of CLC-1011 because this drug will be very valuable for patients with a chronic disease or condition associated with impaired tissue or organ perfusion, in particular, in patients with diabetic ulcers or PAD.

### Conclusion

The above-mentioned results clearly show that CLC-1011 has superior vasodilatory properties. The results also give evidence of the pro-angiogenic and pro-arteriogenic role of CLC-1011. This evidence is based on the shown stimulation of release of pro-angiogenic cytokines, increased capillary density and tissue remodeling in vivo.

1. A method for treatment or prophylaxis of a chronic disease or condition associated with impaired tissue or organ perfusion, comprising administering a therapeutically effective amount of an amino-C₂-C₆-alkyl nitrate or a pharmaceutically acceptable salt thereof to a patient in need thereof.

2. The method according to claim 1, wherein the disease or condition associated with impaired tissue or organ perfusion is a micro- and/or macro-vascular disease.

3. The method according to claim 1, wherein the amino-C₂-C₆-alkyl nitrate or a pharmaceutically acceptable salt thereof induces neo-angiogenesis.

4. The method according to claim 1, wherein the disease or condition associated with impaired tissue and/or organ perfusion is caused by impaired capillary function, impaired muscular function, impaired skin function, impaired cartilage function, impaired myocardial function or impaired retinal function.

5. The method according to claim 1, wherein the disease or condition associated with impaired tissue and/or organ perfusion is caused by impairment of the regeneration of capillaries, arterioles or other vascular tissue.

6. The method according to claim 1, wherein the disease or condition associated with impaired tissue and/or organ perfusion is selected from the group of diseases associated with impaired wound healing, ulcers, and specifically severe peripheral artery disease.

7. The method according to claim 1, wherein the disease or condition is peripheral artery disease or diabetic foot ulcer.

8. The method according to claim 1, wherein the amino-C₂-C₆-alkyl nitrate is an amino-C₂-C₄-alkyl nitrate selected from the group consisting of 4-aminobutyl nitrate, 3-aminopropyl nitrate, 2-amino-1-methylethyl nitrate, 2-aminoethyl nitrate, and pharmaceutically acceptable salts thereof.

9. The method according to claim 1, wherein the amino-C₂-C₆-alkyl nitrate is 2-aminoethyl nitrate or its tosylate salt.

10. The method according to claim 1, wherein the amino-C₂-C₆-alkyl nitrate or pharmaceutically acceptable salt thereof is administered to the patient in need thereof in the form of an extended release composition.

11. The method according to claim 10, wherein the extended release composition releases the amino-C₂-C₆-alkyl nitrate or pharmaceutically acceptable salt thereof over a time period selected from the group consisting of up to 24 hours, 4 to 24 hours, 6 to 24 hours, and 12 to 24 hours.

12. The method according to claim 1, wherein the amino-C₂-C₆-alkyl nitrate or pharmaceutically acceptable salt thereof is administered to the patient in need thereof in an oral dosage form, wherein the amino-C₂-C₆-alkyl nitrate or pharmaceutically acceptable salt thereof is embedded in a non-ionic polymer matrix and optionally coated.

13. The method according to claim 10, wherein the extended release composition is in the form of multiparticulates.

14. The method according to claim 10, wherein the extended release composition is in the form of multiparticulates filled into hard capsules or compressed to a tablet.

15. The method according to claim 1, wherein the amino-C₂-C₆-alkyl nitrate or pharmaceutically acceptable salt thereof is administered to the patient in need thereof in the form of an osmotic drug delivery system.

16. The method according to claim 1, wherein the amino-C₂-C₆-alkyl nitrate or pharmaceutically acceptable salt thereof is administered to the patient in need thereof in the form of a transdermal patch.

17. The method according to claim 1, wherein the amino-C₂-C₆-alkyl nitrate or pharmaceutically acceptable salt thereof is administered to the patient in need thereof in the form of a drug-in-adhesive patch.

18. The method according to claim 1, wherein the amino-C₂-C₆-alkyl nitrate or pharmaceutically acceptable salt thereof is administered to the patient in need thereof in the form of a reservoir patch.

19. A pharmaceutical composition comprising an amino-C₂-C₆-alkyl nitrate or a pharmaceutically acceptable salt thereof and a pharmaceutically acceptable excipient.

20. (canceled)

21. (canceled)

22. The method according to claim 1, wherein the disease or condition involves impaired tissue or organ perfusion or micro-perfusion caused by NO-deficiency.

23. The method according to claim 1, wherein the disease or condition is selected from the group consisting of peripheral artery disease, angina pectoris, type 2 diabetes mellitus, diabetic foot ulcer, pulmonary artery hypertension, congestive heart failure, erectile dysfunction, burn injuries, Moyamoya disease and vascular dysfunction due to a metabolic disease.

24. The methodb according to claim 1, wherein the disease or condition is selected from the group of peripheral artery disease, diabetic foot ulcer and congestive heart failure.

25. (canceled)
